(19) United States
(12) Patent Application Publication
(10) Pub. No.: US 2018/0148781 A1
(43) Pub. Date: May 31, 2018

(54) Antioxidant Compounds For Cleave Formulations That Support Long Reads In Sequencing-By-Synthesis

(71) Applicant: QIAGEN Waltham, Inc., Waltham, MA (US)

(72) Inventors: Luisa Andruzzi, Concord, MA (US); James J. DiMeo, Needham, MA (US); Elizabeth Carpenter, Hudson, MA (US); Visalakshi Meyyappan, Ashland, MA (US)

(21) Appl. No.: 15/799,076

(22) Filed: Oct. 31, 2017

Related U.S. Application Data
(60) Provisional application No. 62/419,674, filed on Nov. 9, 2016.

(51) Int. Cl. C12Q 1/6874

(52) U.S. Cl. CPC C12Q 1/6874

(57) ABSTRACT

Methods, compositions, devices, systems and kits are described including, without limitation, reagents and mixtures for determining the identity of nucleic acids in nucleotide sequences using, for example, sequencing by synthesis methods. Higher base reads with lower error rates are achieved with the use of Ascorbic Acid (AA), also known as Vitamin C, as an antioxidant additive to the cleave reagent.

### FIELD OF THE INVENTION

The invention relates to methods, compositions, devices, systems and kits as described including, without limitation, reagents and mixtures for determining the identity of nucleic acids in nucleotide sequences using, for example, sequencing by synthesis methods.

### BACKGROUND

Over the past 25 years, the amount of DNA sequence information that has been generated and deposited into Genbank has grown exponentially. Traditional sequencing methods (e.g., for example Sanger sequencing) are being replaced by next-generation sequencing technologies that use a form of sequencing by synthesis (SBS), wherein specially designed nucleotides and DNA polymerases are used to read the sequence of DNA templates in a controlled manner. To attain high throughput, many millions of such template spots are arrayed across a sequencing chip and their sequence is independently read out and recorded.

Systems for using arrays for DNA sequencing are known (e.g., Ju et al., U.S. Pat. No. 6,664,079). However, there is a continued need for methods and compositions for increasing the accuracy and/or efficiency of sequencing nucleic acid sequences and increasing the read lengths available for automated sequencing.

### SUMMARY OF THE INVENTION

The invention relates to methods, compositions, devices, systems and kits as described including, without limitation, reagents and mixtures for determining the identity of nucleic acids in nucleotide sequences using, for example, sequencing by synthesis methods. In one embodiment, the present invention contemplates the use of Ascorbic Acid (AA), also known as Vitamin C, as an antioxidant additive to a cleave reagent during the cleave step in sequencing by synthesis (SBS). Such method leads to significant improvement of the sequencing performance (raw error rate thus supporting longer read length). Such effect may be due to enhanced efficacy of the cleave reaction via scavenging of radical by-products and deactivation of excess cleave reagent. Such compounds may build up in the flow cell leading to carry over into the subsequent extension step thus causing premature de-protection of the 3′-OH moiety and impairing single base incorporation rate.

The present invention contemplates, in one embodiment, a method of incorporating labeled nucleotides, comprising: a) providing i) a plurality of nucleic acid primers and template molecules, ii) a polymerase, iii) a cleave reagent comprising a reducing agent and ascorbic acid, and iv) a plurality of nucleotide analogues wherein at least a portion of said nucleotide analogues is labeled with a label attached through a cleavable linker (e.g. a disulfide linker) to the base; b) hybridizing at least a portion of said primers to at least a portion of said template molecules so as to create hybridized primers; c) incorporating a first labeled nucleotide analogue with said polymerase into at least a portion of said hybridized primers so as to create extended primers comprising an incorporated labeled nucleotide analogue; d) detecting said incorporated labeled nucleotide analogue; and e) cleaving the cleavable linker of said incorporated nucleotide analogues with said cleave reagent. In one embodiment, the method is carried out such that read lengths are improved (in comparison to the same method without ascorbic acid). In one embodiment, the method is carried out such that error rates are reduced (in comparison to the same method without ascorbic acid). It is not intended that the present invention be limited to any specific reducing agent. In one embodiment, said reducing agent of said cleave reagent comprises TCEP (tris(2-carboxyethyl)phosphine). In one embodiment, said incorporated nucleotide analogues of step c) further comprise a removable chemical moiety capping the 3′-OH group. In one embodiment, the cleaving of step e) removes the removable chemical moiety capping the 3′-OH group. In one embodiment, the method further comprises f) incorporating a second nucleotide analogue with said polymerase into at least a portion of said extended primers. In one embodiment, the nucleotide analogues are deoxynucleoside triphosphates comprising a 3′-0 position capped by a group comprising methylenedisulfide as a cleavable protecting group and a detectable label reversibly connected to the nucleobase of said deoxynucleoside. In one embodiment, the present invention contemplates a nucleotide analogue with a reversible protecting group comprising methylenedisulfide and a cleavable oxymethylenedisulfide linker between the label and nucleobase.

It is not intended that the present invention be limited to the type of label. A variety of labels are contemplated. In a preferred embodiment, said label is fluorescent.

The present invention also contemplates compositions and reagents. In one embodiment, the present invention contemplates a cleave reagent comprising i) a reducing agent, and ii) ascorbic acid. In one embodiment, said reducing agent is TCEP Tris(2-carboxyethyl)phosphine).

The present invention also contemplates kits, where reagents are supplied with instructions for their use. In one embodiment, the present invention contemplates a kit, comprising i) the cleave reagent and ii) a plurality of nucleotide analogues wherein at least a portion of said nucleotide analogues is labeled with a label attached through a cleavable linker (e.g. a disulfide linker) to the base. In one embodiment, the cleave reagent comprises i) a reducing agent, and ii) ascorbic acid. In one embodiment, said reducing agent is TCEP Tris(2-carboxyethyl)phosphine).

The present invention also contemplates systems, such as systems with flow cells where the flow cells are linked to sources of reagents. See e.g. U.S. Pat. No. 9,145,589, hereby incorporated by reference. In one embodiment, the present invention contemplates a system comprising primers hybridized to template in solution, said solution comprising a cleave reagent, the cleave reagent comprising i) a reducing agent, and ii) ascorbic acid. In one embodiment, said reducing agent is TCEP Tris(2-carboxyethyl)phosphine). In one embodiment, said hybridized primers and template are immobilized. In one embodiment, said hybridized primers and template are in a flow cell.

### DESCRIPTION OF THE INVENTION

A key step in the sequencing-by-synthesis workflow is the removal of the fluorescent label which is covalently attached via a cleavable linker molecule to the ring-position of the heterocyclic base of the nucleotide (reversible terminator) involved in the incorporation step. The efficacy of the cleave step is reflected not only in the efficiency of the fluorescent label cleavage but also in the mitigation of reaction by-products that could accumulate in the flow cell and interfere with subsequent base incorporation step. Examples of such compounds are radical by-products that may form due to radical pathways involved in the omolytic scission of the linker molecule to release the fluorescent label and excess cleave reagent (i. e., tris(2-carboxyethyl)phosphine or TCEP). These may build up in the flow cell and carry over into the subsequent base extension step thus causing premature de-protection of the 3′-OH moiety and causing more than one base to incorporate. An effective cleave step is important for single nucleotide incorporation throughout the sequencing reaction, as well as a prerequisite for low error rate and long read length. To improve the efficacy of the cleave step, molecules that quench radical pathways and oxidize excess TCEP are contemplated, such as ascorbic acid, so as to enhance the efficacy of this reactive step.

Ascorbic Acid (AscH2), also known as Vitamin C, is an essential water-soluble Vitamin, well known for its antiscorbutic and antioxidant functions in humans. It was first identified by virtue of the essential role played in Collagen modification, preventing the nutritional deficiency Scurvy. Vitamin C acts as a co-factor for P4H, i.e. prolyl hydroxylase enzymes, which post-translationally modify collagen and increase the strength and elasticity of tissues. Other roles of ascorbic acid entail reduction of metal ion prosthetic groups of many enzymes, thereby maintaining enzyme activity (Ref. 6). At pH 7.4, 99.95% of vitamin C is present in its mono-anionic form as ascorbate as shown below.

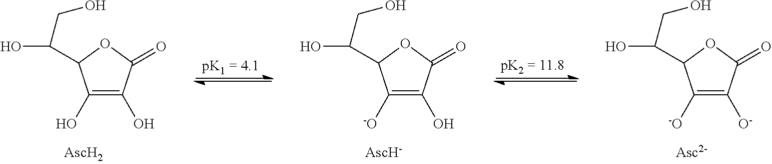

Vitamin C displays anti-oxidant properties in the ascorbate form as represented below:

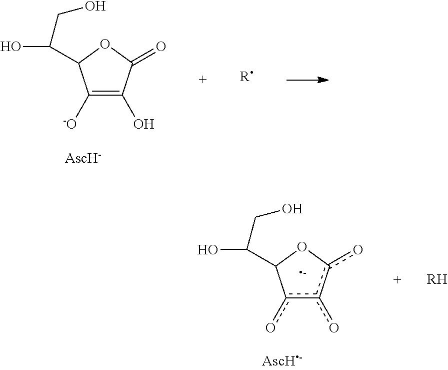

AscH— donates a hydrogen atom (H. or H++e−) to an oxidizing radical to produce the resonance-stabilized tricarbonyl ascorbate free radical. AscH. has a pKa of −0.86; thus, it is not protonated in biology and will be present as Asc.−.

As an electron donor and a redoxactive compound, Vitamin-C participates as a cofactor in many enzymatic reactions, including the hydroxylation of Collagen, synthesis of Carnitine, metabolism of Tyrosine, Nitric Oxide synthesis, Catecholamine synthesis and Peptide Amidation. As a powerful antioxidant Vitamin-C participates in many non-enzymatic reactions preventing oxidation of low-density lipoproteins to alleviate pathological conditions.

Ascorbic acid is especially stable at low pH in the presence of TCEP as a stabilizing agent. While not intending to limit the invention to any particular mechanism, it is believed that TCEP will reduce any dehydro ascorbic acid (DHA) by-product that may form upon oxidation of

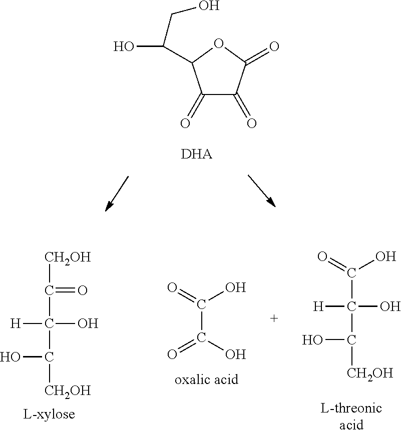

ascorbic acid in the presence of air or other oxidizing conditions, thus preventing degradation pathways that lead to decomposition of DHA to xylose, oxalic acid and threonic acid as outlined in the scheme above.

Due to its antioxidant properties coupled with its mild chemical nature, ascorbic acid has been evaluated for enhancement of cleave reagent performance in the development of long read length sequencing workflow (e.g. average read length >80 bp, and more preferably >100 bp). While not intending to limit the invention to any particular mechanism, it is believed that the cleave reaction step involves omolytic cleavage of a di-sulfide bond in the linker arm off of the ring-position of the heterocyclic base with release of the fluorescent label from the incorporating nucleotide. During this reaction radical species may form and their build up in the flow cell may impair efficiency of the next incorporation cycle. TCEP residuals may also build up in the flow cell. Using an effective antioxidant with redox activity such as ascorbic acid during the cleave reaction may quench these active species, thus preventing premature do-protection of the 3′-OH in the subsequent extension step. This will beneficially impact error rate and read length.

Ascorbic acid has been tested for solubility and stability in cleave reagent formulation. It has been found to be highly soluble over a range of concentrations and stable against precipitation upon prolonged storage at room temperature. Storage of the cleave reagent is typically at −20 C.

Ascorbic acid has been tested for longer read length sequencing performance (150 bp) This early feasibility study entailed 137 cycle and 157 cycle sequencing and head-to-head comparison of ascorbic acid with indole propionic acid (IPA) as a performance benchmarks and Additive-free Cleave as a reference. This study was performed using three different Gene Readers and two types of DNA libraries, i.e. the NA12878/101X gene panel and the PhiX shot gun library. Sequencing metrics were analyzed to provide both system and application key performance indicators (KPIs), e. g., raw error rate, average read length, output (Gb) and false positive rate. Results are shown in the figures and tables.

For example, Table 1 shows the sequencing results where two additives were evaluated in the Cleave reagent, against an additive-free sequencing run using the NA12878/101X gene panel as template. Table 2 shows the sequencing results using the PhiX shot gun library as template.

While all additives were found to outperform additive-free cleave reagent, ascorbic acid was found capable of delivering an average read length of 100-115 bp, filtered according to desired quality score. This outcome represents a 20% improvement over the IPA configuration. Ascorbic acid will permit 125-150 bp target read lengths when optimized for cleave chemical composition and formulation, DNA library size and sequencing protocol. Full compatibility with instrument hardware is also expected for ascorbic acid as observed from inspection of both instrument and liquid waste at the end of sequencing.

### Definitions

To facilitate the understanding of this invention, a number of teams are defined below.

Terms defined herein have meanings as commonly understood by a person of ordinary skill in the areas relevant to the present invention. Terms such as “a”, “an” and “the” are not intended to refer to only a singular entity but also plural entities and also includes the general class of which a specific example may be used for illustration. The terminology herein is used to describe specific embodiments of the invention, but their usage does not delimit the invention, except as outlined in the claims.

The term “about” as used herein, in the context of any of any assay measurements refers to +/−5% of a given measurement

The term “linker” as used herein, refers to any molecule capable of attaching a label and/or chemical moiety that is susceptible to cleavage that may produce toxic radical products. For example, a linker may include, but is not limited to, a disulfide linker and/or an azide linker.

The term “attached” as used herein, refers to any interaction between a first molecule (e.g., for example, a nucleic acid) and a second molecule (e.g., for example, a label molecule). Attachment may be reversible or irreversible. Such attachment includes, but is not limited to, covalent bonding, ionic bonding, Van der Weals forces or friction, and the like.

“Nucleic acid sequence” and “nucleotide sequence” as used herein refer to an oligonucleotide or polynucleotide, and fragments or portions thereof; and to DNA or RNA of genomic or synthetic origin which may be single- or double-stranded, and represent the sense or antisense strand. Such nucleic acids may include, but are not limited to, cDNA, mRNA or other nucleic acid sequences.

The term “an isolated nucleic acid”, as used herein, refers to any nucleic acid molecule that has been removed from its natural state (e.g., removed from a cell and is, in a preferred embodiment, free of other genomic nucleic acid).

In some embodiments, the present invention contemplates hybridizing nucleic acid together. This requires some degree of complementarity. As used herein, the terms “complementary” or “complementarity” are used in reference to “polynucleotides” and “oligonucleotides” (which are interchangeable terms that refer to a sequence of nucleotides) related by the base-pairing rules. For example, the sequence “C-A-G-T,” is complementary to the sequence “O-T-C-A.” Complementarity can be “partial” or “total.” “Partial” complementarity is where one or more nucleic acid bases is not matched according to the base pairing rules. “Total” or “complete” complementarity between nucleic acids is where each and every nucleic acid base is matched with another base under the base pairing rules. The degree of complementarity between nucleic acid strands has significant effects on the efficiency and strength of hybridization between nucleic acid strands. This is of particular importance in amplification reactions, as well as detection methods which depend upon binding between nucleic acids.

The terms “homology” and “homologous” as used herein in reference to nucleotide sequences refer to a degree of complementarity with other nucleotide sequences. There may be partial homology or complete homology (i.e., identity). A nucleotide sequence which is partially complementary, i.e., “substantially homologous,” to a nucleic acid sequence is one that at least partially inhibits a completely complementary sequence from hybridizing to a target nucleic acid sequence. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous sequence to a target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target sequence which lacks even a partial degree of complementarity (e.g., less than about 30% identity); in the absence of non-specific binding the probe will not hybridize to the second non-complementary target.

Low stringency conditions comprise conditions equivalent to binding or hybridization at 42° C. in a solution consisting of 5×SSPE (43.8 g/l NaCl, 6.9 g/l NaH2PO4.H2O and 1.85 g/l EDTA, pH adjusted to 7.4 with NaOH), 0.1% SDS, 5×Denhardt's reagent {50×Denhardt's contains per 500 ml: 5 g Ficoll (Type 400, Pharmacia), 5 g BSA (Fraction V; Sigma)} and 100 μg/ml denatured salmon sperm DNA followed by washing in a solution comprising 5×SSPE, 0.1% SDS at 42° C. when a probe of about 500 nucleotides in length. is employed. Numerous equivalent conditions may also be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol), as well as components of the hybridization solution may be varied to generate conditions of low stringency hybridization different from, but equivalent to, the above listed conditions. In addition, conditions which promote hybridization under conditions of high stringency (e.g., increasing the temperature of the hybridization and/or wash steps, the use of formamide in the hybridization solution, etc.) may also be used.

As used herein, the term “hybridization” is used in reference to the pairing of complementary nucleic acids using any process by which a strand of nucleic acid joins with a complementary strand through base pairing to form a hybridization complex. Hybridization and the strength of hybridization (i.e., the strength of the association between the nucleic acids) is impacted by such factors as the degree of complementarity between the nucleic acids, stringency of the conditions involved, the Tm of the formed hybrid, and the G:C ratio within the nucleic acids.

As used herein the term “hybridization complex” refers to a complex formed between two nucleic acid sequences by virtue of the formation of hydrogen bounds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., C0 t or R0 t analysis) or between one nucleic acid sequence present in solution and another nucleic acid sequence immobilized to a solid support (e.g., a nylon membrane or a nitrocellulose filter as employed in Southern and Northern blotting, dot blotting or a glass slide as employed in in situ hybridization, including FISH (fluorescent in situ hybridization)).

As used herein, the term “Tm” is used in reference to the “melting temperature.” The melting temperature is the temperature at which a population of double-stranded nucleic acid molecules becomes half dissociated into single strands. As indicated by standard references, a simple estimate of the Tm value may be calculated by the equation: Tm=81.5+0.41 (% G+C), when a nucleic acid is in aqueous solution at 1M NaCl. Anderson et al., “Quantitative Filter Hybridization” In: Nucleic Acid Hybridization (1985). More sophisticated computations take structural, as well as sequence characteristics, into account for the calculation of Tm.

As used herein the term “stringency” is used in reference to the conditions of temperature, ionic strength, and the presence of other compounds such as organic solvents, under which nucleic acid hybridizations are conducted. “Stringency” typically occurs in a range from about Tm to about 20° C. to 25° C. below Tm. A “stringent hybridization” can be used to identify or detect identical polynucleotide sequences or to identify or detect similar or related polynucleotide sequences. For example, when fragments are employed in hybridization reactions under stringent conditions the hybridization of fragments which contain unique sequences (i.e., regions which are either non-homologous to or which contain less than about 50% homology or complementarity) are favored. Alternatively, when conditions of “weak” or “low” stringency are used hybridization may occur with nucleic acids that are derived from organisms that are genetically diverse (i.e., for example, the frequency of complementary sequences is usually low between such organisms).

As used herein, the term “amplifiable nucleic acid” is used in reference to nucleic acids which may be amplified by any amplification method. It is contemplated that “amplifiable nucleic acid” will usually comprise “sample template.”

As used herein, the term “sample template” or (more simply) “template” refers to nucleic acid originating from a sample which is analyzed for the presence of a target sequence of interest. In contrast, “background template” is used in reference to nucleic acid other than sample template which may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

“Amplification” is defined as the production of additional copies of a nucleic acid sequence and is generally carried out using polymerase chain reaction. Dieffenbach C. W. and G. S. Dvcksler (1995) In: PCR Primer, a Laboratory Manual, Cold Spring Harbor Press, Plainview, N.Y.

The DNA sequence is determined by multiple cycles of chemistry on an instrument. While a variety of sequencing instruments can be used (Applied Biosystems SOLiD System, Illumina Next-Generation Sequencing Platforms, OLiD/Ion Torrent PGM from Life Sciences, Genome Analyzer/HiSeq 2000/MiSeq from Illumina, and GS FLX Titanium/OS Junior from Roche, Ion Proton™ System from ThermoFisher Scientific, QIAGEN's GeneReader DNA sequencing system, and other next generation sequencers), a preferred instrument is QIAGEN's GeneReader DNA sequencing system. In one embodiment, a DNA sequence is determined by a method of sequencing by synthesis (SBS). In one embodiment, each cycle of sequencing consists of eight steps: extension 1, extension 2, wash 1, addition imaging solution, imaging, wash 2, cleave, and wash 3. Data collected during imaging cycles is processed by analysis software yielding error rates, throughput values, and applied phasing correction values.

As used herein, the term “polymerase chain reaction” (“PCR”) refers to the method of K. B. Mullis U.S. Pat. Nos. 4,683,195 and 4,683,202, herein incorporated by reference, which describe a method for increasing the concentration of a segment of a target sequence in a mixture of genomic DNA without cloning or purification. The length of the amplified segment of the desired target sequence is determined by the relative positions of two oligonucleotide primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”. With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of 32P-labeled deoxynucleotide triphosphates, such as dCTP or dATP, into the amplified segment). In addition to genomic DNA, any oligonucleotide sequence can be amplified with the appropriate set of primer molecules. In particular, the amplified segments created by the PCR process itself are, themselves, efficient templates for subsequent PCR amplifications.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxy-ribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers; to an oligonucleotide (i.e., a sequence of nucleotides), whether occurring naturally as in a purified restriction digest or produced synthetically, recombinantly or by PCR amplification, which is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

The term “label” or “detectable label” are used herein, to refer to any composition detectable by spectroscopic, photochemical, biochemical, immunochemical, electrical, optical or chemical means. Such labels include biotin for staining with labeled streptavidin conjugate, magnetic beads (e.g., Dynabeads®), fluorescent dyes (e.g., fluorescin, texas red, rhodamine, green fluorescent protein, and the like), radiolabels (e.g., 3H, 125I, 35S, 14C, or 32P), enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads. Patents teaching the use of such labels include, but are not limited to, U.S. Pat. Nos. 3,817,837; 3,850,752; 3,939,350; 3,996,345; 4,277,437; 4,275,149; and 4,366,241 (all herein incorporated by reference).

In a preferred embodiment, the label is typically fluorescent and is linked to the base of the nucleotide. For cytosine and thymine, the attachment is usually to the 5-position. For the other bases, a deaza derivative is created and the label (e.g. sulfo-Cy5) is linked to a 7-position of deaza-adenine or deaza-guanine.

The labels contemplated in the present invention may be detected by many methods. For example, radiolabels may be detected using photographic film or scintillation counters, fluorescent markers may be detected using a photodetector to detect emitted light Enzymatic labels are typically detected by providing the enzyme with a substrate and detecting, the reaction product produced by the action of the enzyme on the substrate, and calorimetric labels are detected by simply visualizing the colored label.

The term “luminescence” and/or “fluorescence”, as used herein, refers to any process of emitting electromagnetic radiation (light) from an object, chemical and/or compound. Luminescence and/or fluorescence results from a system which is “relaxing” from an excited state to a lower state with a corresponding release of energy in the form of a photon. These states can be electronic, vibronic, rotational, or any combination of the three. The transition responsible for luminescence can be stimulated through the release of energy stored in the system chemically or added to the system from an external source. The external source of energy can be of a variety of types including, but not limited to, chemical, thermal, electrical, magnetic, electromagnetic, physical or any other type capable of causing a system to be excited into a state higher than the ground state. For example, a system can be excited by absorbing a photon of light, by being placed in an electrical field, or through a chemical oxidation-reduction reaction. The energy of the photons emitted during luminescence can be in a range from low-energy microwave radiation to high-energy x-ray radiation. Typically, luminescence refers to photons in the range from UV to IR radiation.

### DESCRIPTION OF THE FIGURES

FIG. 1 is a graph comparing ascorbic acid read length distribution (red) against IPA (green) along with an additive-free control (No Add) (purple) results generated in a sequencing run using the NA12878/101X gene panel as template.

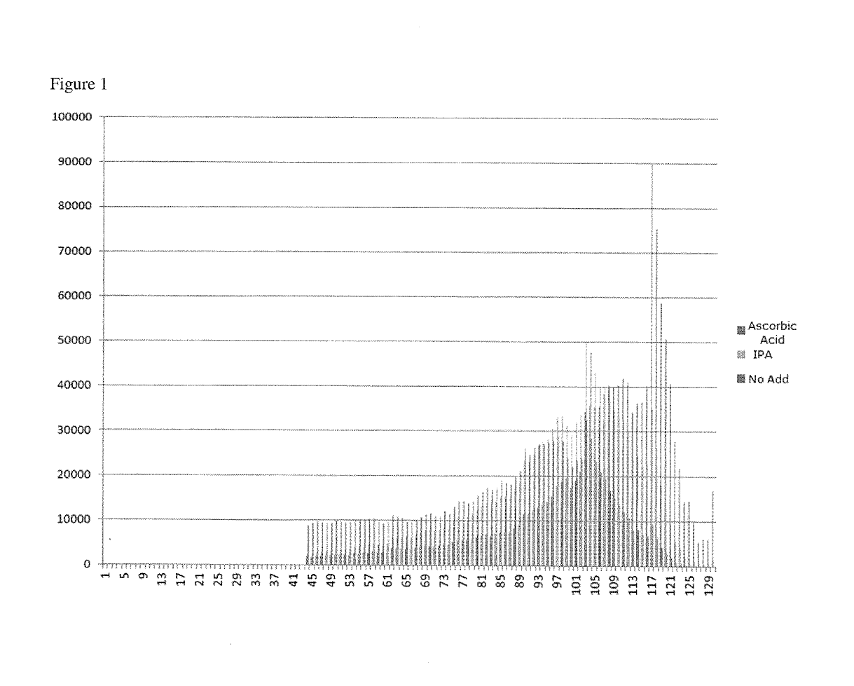

FIG. 2 is a graph comparing the raw error rate for ascorbic acid and IPA along with an additive-free control (No add) where the results were generated in a sequencing run using the NA12878/101X gene panel as template. The raw error rate for ascorbic acid is significantly better than i) the no additive run, and ii) the run with IPA.

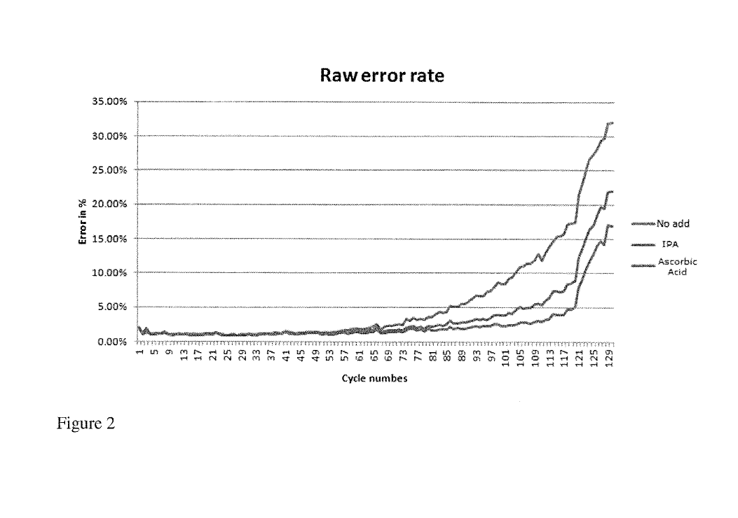

FIGS. 3A, 3B, 3C and 3D are graphs comparing the signal margin using labeled C, T, A and G (respectively) where the Cleave reagent either had ascorbic acid or IPA along with an additive-free control (No add—see arrow) in a sequencing run. The Alexa dye was used to label C; the Cy3 dye was used to label T; the Rox dye was used to label A; the Cy5 dye was used to label G. Additive Cleave chemistry produces a signal margin that is significantly better than no additive, with ascorbic acid delivering the highest signal margin among all additives.

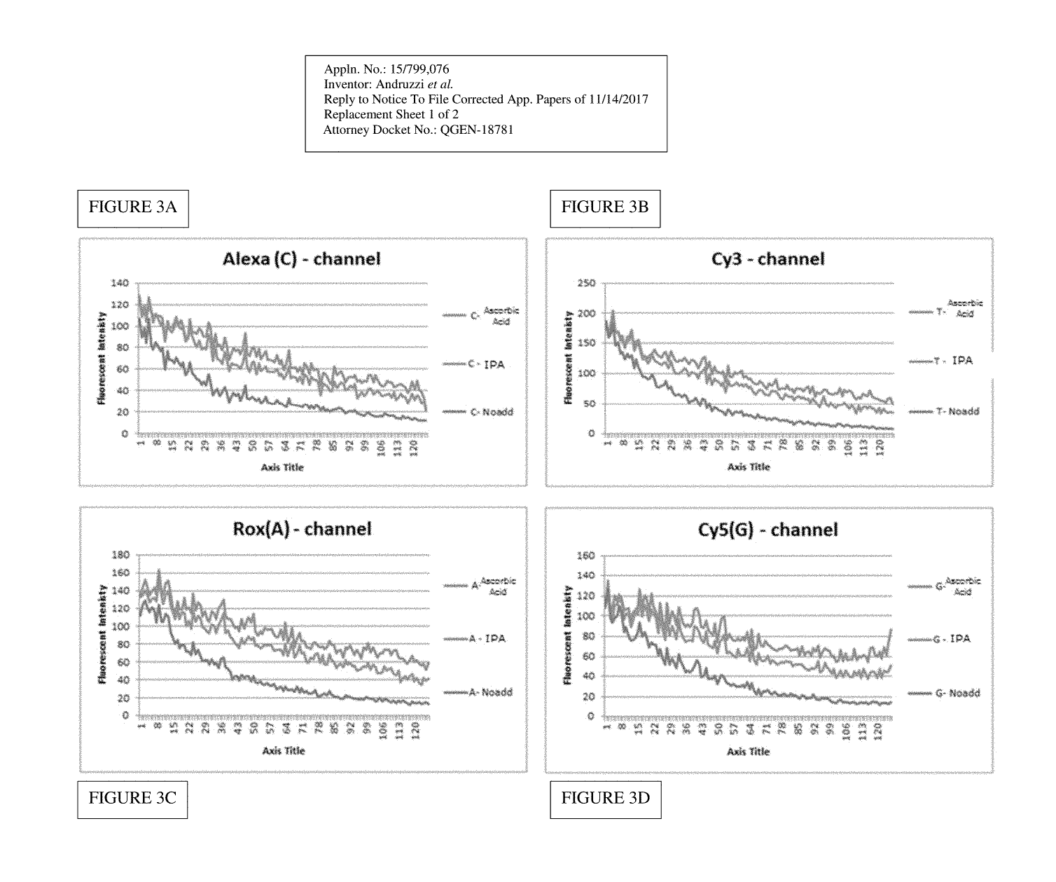

FIGS. 4A, 4B and 4C are perfect read heat maps generated with an additive-free control (No Additive), IPA and ascorbic acid in a sequencing run, respectively. The results show that additive cleave chemistry solves the “race track” issue historically observed with no additive cleave. All additives appear to be equally effective in erasing the race track.

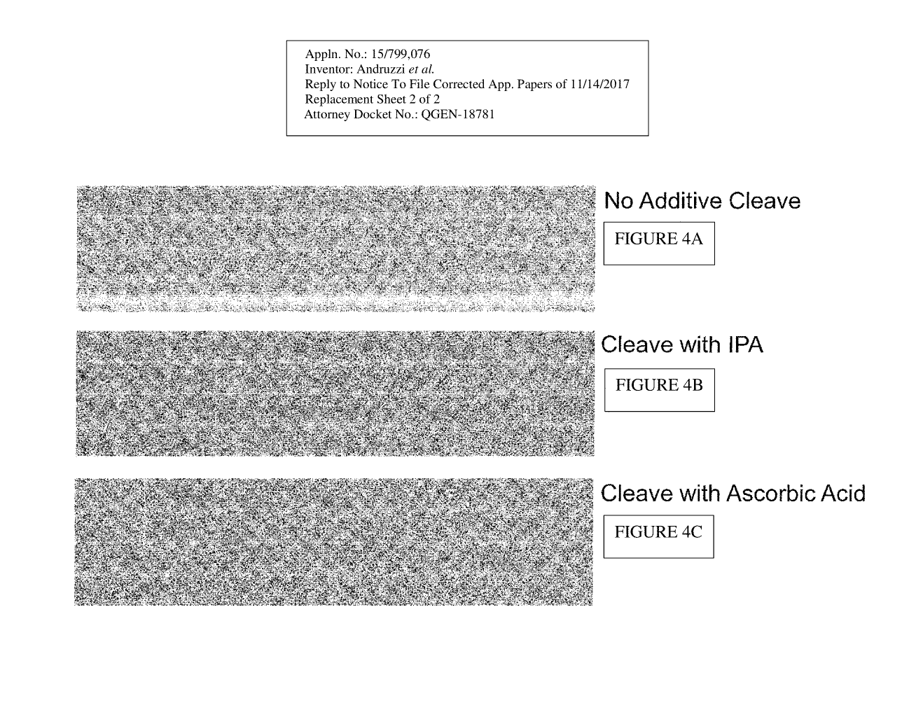

FIG. 5 is a graph comparing ascorbic acid read length distribution against IPA results generated in a sequencing run using the PhiX shot gun library as template.

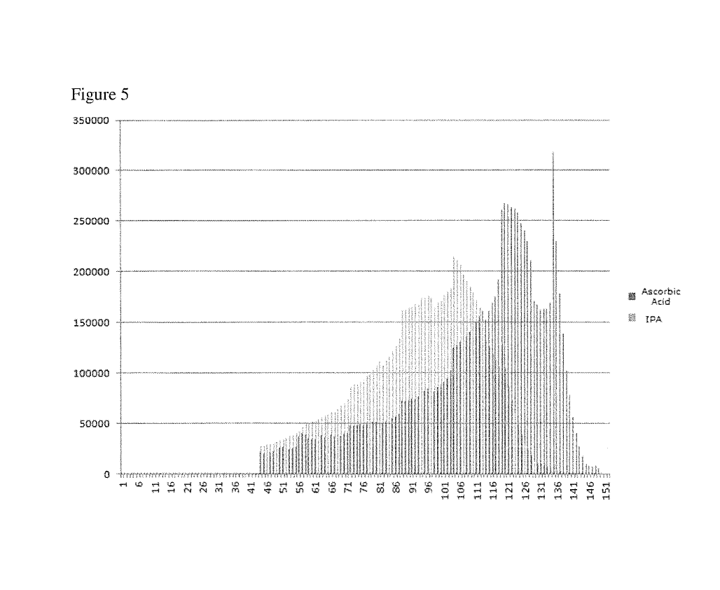

### DETAILED DESCRIPTION OF THE INVENTION

In one embodiment, the present invention contemplates a series of method steps performed by an automated sequencing by synthesis instrument. See U.S. Pat. No. 9,145,589, hereby incorporated by reference. In one embodiment, the instrument is comprised of numerous reagent reservoirs. Each reagent reservoir has a specific reactivity reagent dispensed within the reservoir to support the SBS process, for example:

One reactive step in a method for sequencing by synthesis using cleavable fluorescent nucleotide reversible terminators comprises cleaving a fluorescent label from a nucleotide analogue molecule. It is not intended that the present invention be limited by the nature of the cleaving agent.

In one embodiment, the SBS method comprises doing different steps at different stations. By way of example, each station is associated with a particular step. While not limited to particular formulations, some examples for these steps and the associated reagents are shown below:

1) Extend A Reagent: Comprises reversibly terminated labeled nucleotides and polymerase. The composition of Extend A is as follows:

| Component | Conc |
|---|---|
| PNSE (% wt/vol) | 0.005% |
| Tris × HCl (pH 8.8), mM | 50 |
| NaCl (mM) | 50 |
| EDTA (mM) | 1 |
| MgSO4 (mM) | 10 |
| Cystamine (mM) | 1 |
| Glycerol (% wt/vol) | 0.01% |
| Therminator IX* (U/ml) | 10 |
| N3-dCTP (μM) | 3.83 |
| N3-dTTP (μM) | 3.61 |
| N3-dATP (μM) | 4.03 |
| N3-dGTP (μM) | 0.4 |
| Alexa488-dCTP (nM) | 550 |
| R6G-dUTP (nM) | 35 |
| ROX-dATP (nM) | 221 |
| Cy5-dGTP (nM) | 66 |

*with Alkylated free Cysteine

2) Extend B Reagent: Comprises reversibly terminated unlabeled nucleotides and polymerase, but lacks labeled nucleotide analogues. The composition of Extend B is as follows:

| Component | Conc |
|---|---|
| PNSE (% wt/vol) | 0.005% |
| Tris × HCl (pH 8.8), mM | 50 |
| NaCl (mM) | 50 |
| EDTA (mM) | 1 |
| MgSO4 (mM) | 10 |
| Glycerol (% wt/vol) | 0.01% |
| Therminator IX* (U/ml) | 10 |
| N3-dCTP (μM) | 21 |
| N3-dTTP (μM) | 17 |
| N3-dATP (μM) | 21 |
| N3-dGTP (μM) | 2 |

*Alkylated free Cysteine

3) Wash solution 1 with a detergent (e.g., polysorbate 20) citrate buffer (e.g., saline)
4) Cleave Reagent: A cleaving solution composition is as follows:

| Component | Conc |
|---|---|
| NaOH (mM) | 237.5 |
| TrisHCl (pH 8.0) (mM) | 237.5 |
| TCEP (mM) | 50 |

5) Wash solution 2 with a detergent (e.g., polysorbate 20) a tris(hydroxymethyl)-aminomethane (Tris) buffer.

### Experimental

### Example 1

In one embodiment, the present invention contemplates a SBS method comprising the steps shown in Table 3. See Olejink et al., “Methods And Compositions For Inhibiting Undesired Cleaving Of Labels” U.S. Pat. No. 8,623,598 (herein incorporated by reference in its entirety).

TABLE 3
An Exemplary SBS Workflow

| | Fluid Movements | | | | | |
|---|---|---|---|---|---|---|
| | | Volume | Speed | Station | Temp | Time |
| Step | Reagent | mL | mL/s | Number | ° C. | [s] |
| 1. Dispense Reagent | Reagent 1 | 100 | 67 | 3 | 65 | 7 |
| 2. Incubate Reagent | Reagent 1 | n/a | n/a | 3 | 65 | 210 |
| 3. Dispense Reagent | Reagent 2 | 100 | 67 | 4 | 65 | 7 |
| 4. Incubate Reagent | Reagent 2 | n/a | n/a | 4 | 65 | 210 |
| 5. Dispense Reagent | Reagent 3 | 330 | 27 | 5 | Ambient | 12 |
| 6. Dispense Reagent | Reagent 4 + 5 | 200 | 27 | 5 | Ambient | 15 |
| 7. Image | n/a | n/a | n/a | 11 | Ambient | 210 |
| 8. Dispense Reagent | Reagent 3 | 330 | 27 | 20 | 65 | 12 |
| 9. Dispense Reagent | Reagent 6 | 100 | 67 | 1 | 65 | 7 |
| 10. Incubate Reagent | Reagent 6 | n/a | n/a | 1 | 65 | 210 |
| 11. Incubate Reagent | Reagent 6 | n/a | n/a | 2 | 65 | 210 |
| 12. Dispense Reagent | Reagent 7 | 990 | 27 | 2 | 65 | 37 |

13. Go to Step 1
Reagent 1 = Extend A;
Reagent 2 = Extend B;
Reagent 3 = Wash;
Reagent 4 = Image A;
Reagent 5 = Image B;
Reagent 6 = Cleave;
and Reagent 7 = Wash 11

### Example 2

In one embodiment, the present invention contemplates a method for Cleave Optimization for Long Reads: Ascorbic Acid Titration. Conditions may vary in this method. In one embodiment, ascorbic acid is approximately 40 mM in Cleave Mix. In one embodiment, ascorbic acid is approximately 60 mM in Cleave Mix. See Table 4. In one embodiment, the method uses Four GeneReaders and same gene panel pool. In one embodiment, the Sequencing protocol comprises 157 cycles.

The condition: 60 mM AddA in Cleave yields improved performance with the average read length ˜10% longer than AA 40 mM and the raw error rate ˜20% lower than AA 40 mM. Condition Locked for System Verification to support GR1.1 Product Launch: AA 60 mM.

Re-analysis with new SW (upgraded with new look-up-table) yielded improved KPI's: Average read length=130 bp; Filtered Output=2.2 Gb; and Q-Score=84%@Q25; See sample data set in Table 5. Table 5 shows Cleave Production Lot: QC by functional test prior to System Verification. Finding: IN-SPEC. AVG Read length 130 bp; Filtered Output >2 Gb; and Filtered Error ˜0.6%.

TABLE 1

| | | | | | | | % | | | | | | | |
|---|---|---|---|---|---|---|---|---|---|---|---|---|---|---|
| | | | | | | % | Bead | Q @ | % @ | Avg | Fil- | % | | |
| Type of | Beads/ | Output | % | % | % | Poly- | reten- | 85% | Q25 | read | tered | Per- | | |
| Additive | tiles | (Mb) | Live | BC | Mapped | clonal | tion | (MFST) | (MFST) | length | error | fect | Lead | Lag |
| Ascorbic | 395125.58 | 1327958543 | 58% | 74% | 37% | 28% | 96% | 24.82 | 85% | 103.359 | 0.524 | 68% | 0.41348 | 0.14564 |
| Acid | 408815.284 | 1369125970 | 54% | 75% | 36% | 29% | 97% | 24.20 | 85% | 103.709 | 0.542 | 67% | 0.3825 | 0.16348 |
| | 405195.42 | 1378585341 | 54% | 75% | 37% | 29% | 95% | 24.48 | 85% | 104.208 | 0.528 | 68% | 0.39217 | 0.16157 |
| Ascorbic | 416758.875 | 1228462334 | 51% | 72% | 32% | 29% | 97% | 24.00 | 85% | 104.49 | 0.587 | 66% | 0.42492 | 0.12226 |
| Acid | 401621.58 | 1283575472 | 54% | 74% | 34% | 28% | 97% | 24.11 | 85% | 105.33 | 0.574 | 66% | 0.36322 | 0.14735 |
| | 417307.261 | 1300734915 | 58% | 74% | 33% | 29% | 97% | 24.00 | 84% | 106.30 | 0.594 | 66% | 0.36172 | 0.14775 |
| IPA | 423407.125 | 1140366855 | 58% | 73% | 32% | 33% | 98% | 24.00 | 84% | 96.20 | 0.637 | 65% | 0.38553 | 0.12845 |
| | 417375.92 | 1138434690 | 56% | 73% | 32% | 33% | 98% | 24.00 | 84% | 97.14 | 0.623 | 65% | 0.38643 | 0.1266 |
| | 427176.91 | 1136807136 | 55% | 73% | 31% | 33% | 97% | 24.00 | 84% | 96.66 | 0.631 | 65% | 0.40926 | 0.12607 |
| No | 429057.307 | 837839705 | 54% | 72% | 27% | 35% | 97% | 24.00 | 83% | 83.47 | 0.689 | 66% | 0.30505 | 0.1009 |
| Additives | 411802.284 | 857618430 | 54% | 72% | 28% | 34% | 97% | 24.00 | 84% | 86.46 | 0.628 | 67% | 0.28894 | 0.12394 |
| | 408721.852 | 895905054 | 55% | 73% | 28% | 34% | 98% | 24.00 | 84% | 87.73 | 0.625 | 67% | 0.30965 | 0.12033 |

TABLE 2

| | | Output | | | | | | % bead | Q @ 85% |
|---|---|---|---|---|---|---|---|---|---|
| Run | GR | (MFST) | Beads/tile | % live | % BC | % mapped | % polyclonal | retention | (MFST) |
| Ascorbic Acid | 8.5 | 1.123258 | 206242.05 | 50% | 71% | 36% | 29% | 95% | 24.0 |
| | | 1.028756 | 313033.35 | 47% | 70% | 33% | 30% | 96% | 24.0 |
| Ascorbic Acid | 8.5 | 1.067198 | 433712.41 | 43% | 67% | 26% | 40% | 97% | 24.0 |
| | | 1.114052 | 431691.83 | 43% | 68% | 27% | 37% | 97% | 24.0 |
| IPA | 8.5 | 0.838 | 419932.48 | 39% | 66% | 24% | 35% | 97% | 24.0 |
| | | 0.817013 | 416846.77 | 38% | 66% | 24% | 35% | 97% | 24.0 |
| | 8.26 | 0.915722 | 389958.05 | 37% | 65% | 28% | 25% | 98% | 24.0 |
| | | 0.9445307 | 399497.89 | 42% | 68% | 28% | 31% | 96% | 24.0 |
| | 8.6 | 0.97005 | 428698.34 | 41% | 68% | 27% | 32% | 97% | 24.0 |
| | | 0.882115 | 419704.92 | 39% | 67% | 27% | 30% | 97% | 24.0 |

| | | % @ Q25 | avg read | error rate | error rate | | | |
|---|---|---|---|---|---|---|---|---|
| Run | GR | (MFST) | length | (MFST) | (MFST) | % perfect | lead | lag |
| Ascorbic Acid | 8.5 | 0.802557 | 113.1077 | 0.008847 | 0.884679 | 62% | 0.419159 | 0.128057 |
| | | 0.811435 | 113.9125 | 0.009683 | 0.968294 | 60% | 0.403841 | 0.187966 |
| Ascorbic Acid | 8.5 | 0.795629 | 104.82 | 0.011809 | 1.180904 | 54% | 0.350568 | 0.268989 |
| | | 0.799917 | 104.50 | 0.0011285 | 1.128478 | 56% | 0.421591 | 0.187352 |
| IPA | 8.5 | 0.802557 | 93.1 | 0.010112 | 1.01123 | 58% | 0.342148 | 0.089227 |
| | | 0.811435 | 94.0 | 0.009678 | 0.967772 | 59% | 0.30392 | 0.130568 |
| | 8.26 | 0.816509 | 96.6 | 0.008427 | 0.842717 | 63% | 0.330466 | 0.105932 |
| | | 0.810365 | 97.7 | 0.009399 | 0.939915 | 60% | 0.307489 | 0.140761 |
| | 8.6 | 0.797105 | 89.3 | 0.009712 | 0.971189 | 59% | 0.31158 | 0.118273 |
| | | 0.798682 | 90.0 | 0.009516 | 0.951585 | 60% | 0.337386 | 0.093807 |

TABLE 4

| Run | | | |
|---|---|---|---|
| Condition | GR | Run date | Run Link |
| 40 mM | 8.34 | Jul. 8, 2016 | \\walisilon01.global.enterprise\ngs\8.34\2016_07_08_155019_GDP4_Clv_40AA_Baseline_NP\FC_1-HIL01_{—} |
| AddA | | | 81300630151049923000000465 |
| Cleave | 8.17 | Jul. 8, 2016 | \\walisilon01.global.enterprise\ngs\8.17\2016_07_08_170434_40aaClv_BRCA_JA\FC_HIL01_{—} |
| | | | 81300630151041611071600845 |
| | 8.2 | Jul. 8, 2016 | \\walisilon01.global.enterprise\ngs\8.2\2016_07_08_155257_GDP4_Clv_40AA_Baseline_NP\FC_1-HIL01_{—} |
| | | | 81300630151049923000000466 |
| | 8.24 | Jul. 8, 2016 | \\walisilon01.global.enterprise\ngs\8.24\2016_07_08_165955_40AAclv_157_BRCA_JA\FC_HIL01_{—} |
| | | | 81300630151041611071600844 |
| | 8.24 | Jul. 29, 2016 | \\walisilon01.global.enterprise\ngs\8.24\2016_07_29_173012_GDP4_40mMAAclv_JA\FC_MG56_{—} |
| | | | 81300630154020789011701222 |
| | 8.26 | Jul. 29, 2016 | \\walisilon01.global.enterprise\ngs\8.26\2016_07_29_171821_GDP4_Clv_40AA_Baseline_NP\FC_1-MG56_{—} |
| | | | 81300630154020789011701081 |
| AVG | | | |
| STD | | | |
| 60 mM | 8.34 | Jul. 11, 2016 | \\walisilon01.global.enterprise\ngs\8.34\2016_07_11_144350_GDP4_Clv_60AA_NP\FC_1-HIL01_{—} |
| AddA | | | 8130063015105000000000237 |
| Cleave | 8.17 | Jul. 11, 2016 | \\walisilon01.global.enterprise\ngs\8.17\2016_07_11_145057_60mMAAclv_BRCA_JA\FC_1_HIL01_{—} |
| | | | 81300630151050000000000170 |
| | 8.2 | Jul. 11, 2016 | \\walisilon01.global.enterprise\ngs\8.2\2016_07_11_144423_GDP4_Clv_60AA_NP\FC_1-HIL01_{—} |
| | | | 81300630151050000000000466 |
| | 8.24 | Jul. 11, 2016 | \\walisilon01.global.enterprise\ngs\8.24\2016_07_11_144926_60mMAAclv_BRCA_JA\FC_1_HIL01_{—} |
| | | | 81300630151050000000000171 |
| | 8.31 | Jul. 29, 2016 | \\walisilon01.global.enterprise\ngs\8.31\2016_07_29_171010_GDP4_Clv_60AA_NP\FC_1-MG56_{—} |
| | | | 81300630154020789011701041 |
| | 8.17 | Jul. 29, 2016 | \\walisilon01.global.enterprise\ngs\8.17\2016_07_29_172720_GDP4_60mMAAclv_JA\FC_MG56_{—} |
| | | | 81300630154020789011701028 |
| AVG | | | |
| STD | | | |

| | | Output | Beads/ | | | % | % poly- | % @ Q25 | | Error Rate | Raw Error | % Per- |
|---|---|---|---|---|---|---|---|---|---|---|---|---|
| Type | Cycles | (MFST) | tile | % live | % BC | mapped | clonal | (MFST) | AVG RL | (MFST) | Rate (FQ) | fect |
| BRCA | 157 | 1986557669 | 402500 | 50.45% | 79.28% | 32.64% | 31.09% | 85.35% | 116.465135 | 0.75% | 2.95% | 59.54% |
| BRCA | 157 | 1865280044 | 401939 | 47.08% | 78.14% | 30.29% | 33.05% | 84.91% | 117.950615 | 0.79% | 2.79% | 59.12% |
| BRCA | 157 | 1940469561 | 413770 | 50.48% | 79.86% | 32.73% | 30.92% | 84.65% | 110.391065 | 0.78% | 3.86% | 58.87% |
| BRCA | 157 | 2004985129 | 420905 | 48.89% | 79.72% | 31.31% | 33.81% | 83.24% | 117.095261 | 0.86% | 2.76% | 55.67% |
| BRCA | 157 | 1893983911 | 420704 | 50.55% | 80.43% | 31.58% | 34.96% | 80.75% | 111.718784 | 0.94% | 3.95% | 51.38% |
| BRCA | 157 | 2116925178 | 431809 | 50.78% | 80.23% | 32.97% | 32.32% | 84.30% | 114.454753 | 0.74% | 2.95% | 59.02% |
| | | | | | | | | | 114.679 | 0.79% | 3.00% | 58% |
| | | | | | | | | | 3.443867719 | 0.00045504 | 0.01 | 0.02 |
| BRCA | 157 | 2195175777 | 427841 | 51.85% | 80.37% | 33.88% | 32.01% | 84.54% | 120.587583 | 0.8% | 2.76% | 59% |
| BRCA | 157 | 2122117645 | 415214 | 48.99% | 79.01% | 31.57% | 33.79% | 85.28% | 124.649223 | 0.75% | 2.27% | 58% |
| BRCA | 157 | 2169462886 | 425978 | 50.15% | 80.07% | 33.29% | 31.33% | 84.64% | 120.767997 | 0.8% | 2.68% | 59% |
| BRCA | 157 | 2170473943 | 421358 | 49.25% | 79.62% | 32.13% | 33.36% | 84.60% | 123.435264 | 0.75% | 2.32% | 57% |
| BRCA | 157 | 2353647961 | 424293 | 51.67% | 80.75% | 34.30% | 31.71% | 85.43% | 124.534612 | 0.8% | 2.18% | 59% |
| BRCA | 157 | 2106601256 | 408760 | 50.41% | 79.42% | 32.22% | 34.08% | 84.49% | 123.141096 | 0.8% | 2.27% | 58% |
| | | | | | | | | | 121.360 | 0.80% | 2.50% | 58% |
| | | | | | | | | | 2.006 | 0.00013273 | 0.002 | 0.009 |

TABLE 5

| | | Loca- | | % Bead | | % | % Per- | % @ | AVG Read | FASTQ | FASTQ |
|---|---|---|---|---|---|---|---|---|---|---|---|
| Date | Run ID | tion | GR | ret | Tiles | Mapped | fect | Q25 | Length | Error | Output |
| Oct. 21, 2016 | CI01_Im04_CIA05_FC982_997 | Hilden | 8.39 | 0.9599996 | 130 | 33% | 61% | 85% | 130.15 | 0.62% | 2.4 Gb |
| Oct. 21, 2016 | CI01_Im04_CIA05_FC982_997 | Hilden | 8.39 | 0.9413901 | 130 | 28% | 61% | 85% | 130.221 | 0.63% | 2.01 Gb |

We claim:

1. A method of incorporating labeled nucleotides, comprising:
a) providing i) a plurality of nucleic acid primers and template molecules, ii) a polymerase, iii) a cleave reagent comprising a reducing agent and ascorbic acid, and iv) a plurality of nucleotide analogues wherein at least a portion of said nucleotide analogues is labeled with a label attached through a cleavable linker to the base;
b) hybridizing at least a portion of said primers to at least a portion of said template molecules so as to create hybridized primers;
c) incorporating a first labeled nucleotide analogue with said polymerase into at least a portion of said hybridized primers so as to create extended primers comprising an incorporated labeled nucleotide analogue;
d) detecting said incorporated labeled nucleotide analogue; and
e) cleaving the cleavable linker of said incorporated nucleotide analogues with said cleave reagent.

2. The method of claim 1, wherein said reducing agent of said cleave reagent comprises TCEP (tris(2-carboxyethyl)phosphine).

3. The method of claim 1, wherein said incorporated nucleotide analogues of step c) further comprise a removable chemical moiety capping the 3′-OH group.

4. The method of claim 3, wherein the cleaving of step e) removes the removable chemical moiety capping the 3′-OH group.

5. The method of claim 4, wherein the method further comprises:
f) incorporating a second nucleotide analogue with said polymerase into at least a portion of said extended primers.

6. The method of claim 1, wherein said label is fluorescent.

7. A cleave reagent comprising i) a reducing agent, and ii) ascorbic acid.

8. The cleave reagent of claim 7, wherein said reducing agent is TCEP Tris(2-carboxyethyl)phosphine).

9. A kit, comprising i) the cleave reagent of claim 7 and ii) a plurality of nucleotide analogues wherein at least a portion of said nucleotide analogues is labeled with a label attached through a cleavable linker to the base.

10. A system comprising primers hybridized to template in solution, said solution comprising the cleave reagent of claim 7.

11. The system of claim 10, wherein said hybridized primers and template are immobilized.

12. The system of claim 11, wherein said hybridized primers and template are in a flow cell.
